(19) United States
(12) Patent Application Publication
(10) Pub. No.: US 2007/0248647 A1
(43) Pub. Date: Oct. 25, 2007

(54) Erythritol suppression of ghrelin

(76) Inventor: Keith Earl Kenyon, Sherman Oaks, CA (US)

Correspondence Address: KEITH E. KENYON, M.D., 4501 NAGLE AVENUE, SHERMAN OAKS, CA, 91423, US

(21) Appl. No.: 11/397,042

(22) Filed: Apr. 3, 2006

(51) Int. Cl. A61K 47/00

(52) U.S. Cl. 424/439

(57) ABSTRACT

The present invention provides novel means to make a new food, especially as a result of cooking or baking but also with cold food preparation to dilute the fat in such foods. This new food is the result of eliminating sugar and independent flour in pastry by mixing erythritol and either dietary protein and dietary polysaccharides or both together with approximately the same weight of water as the combined solids and then incorporating this mass with other ingredients in a batter to make pastry when subjected to heat. In this way the ghrelin-suppressing action of erythritol in the small intestine will be concentrated the most effectively for maximum effect.

### RELATED DISCLOSURES

This disclosure is related to patent application Ser. No. 11/301,181, “Z-Trim Combined Directly with Erythritol”, 11/358,619, “Binding Virtually Zero Calories Saccharide-Type Bulk to Dietary Protein” and 11/371,281, “Soy-Erythritol Compositions for Health”.

### STATEMENT REGARDING FEDERALLY SPONSORED RESEARCH/DEVELOPMENT

The present invention does not involve any form of federally sponsored research or development.

### FIELD OF THE INVENTION

The present invention relates to erythritol formulations used in baking for weight management, all containing substantial amounts of erythritol combined with protein and/or polysaccharides to reduce overall calories and suppress ghrelin.

### BACKGROUND OF THE INVENTION

This disclosure is being provided to address the newly discovered weight management property of the sugar alcohol, erythritol, first disclosed in patent application Ser. No. 11/371,281, “Soy-Erythritol Compositions for Health”, as it applies now to maximum suppression of ghrelin. Erythritol has the physical chemical property of being both endothermic and exothermic depending on the energy level it is exposed to. When water is added to it at room temperature it exhibits a net negative or endothermic heat of solution property, and the water-erythritol combination turns cold. On the other hand, when heated with water and then allowed to cool, it will precipitate out yet remain hot much longer than would be expected as it cools down. This combination of properties renders it a poor sweetener when used in baking unlike sucrose which stays in solution. As a consequence, those skilled in the art have used other sugars to complement erythritol by dilution such as inulin, including FOS (fructose oligosaccharide), as well as maltodextrin, tagatose and even sucrose. Unfortunately this dilutes the appetite-suppression action of erythritol also, so renders it much less effective in weight management. The problem is that the effective weight management property of erythritol requires a large volume of the ingredient to be deposited together in the small intestine where it ultimately is absorbed to affect the differential osmolarity in order to suppress ghrelin. It would seem apparent that the more erythritol there is concentrated in the small intestine at a given time, the more the substance can affect appetite suppression. The fact that the conventional use for erythritol is that of a sweetener means that it is ineffective for the purpose of this disclosure because it is rendered in small amounts and reaches the small intestine in very dilute so ineffective concentrations.

This disclosure seeks to enable the maximum amount of erythritol to enter the small intestine in the smallest volume possible so as to enable maximum absorption of the erythritol in the shortest sequence of time. In this way the greatest shift in osmolarity can be accomplished so as to render the maximum suppression of ghrelin possible in this short time period. It would appear that combining erythritol with one or more of a number of ingredients that it can bind to, in part as disclosed in patent application Ser. Nos. 11/301,181, 11/358,619 and 11/371,281, would be necessary to accomplish this. Binding is required to be done as disclosed in the aforementioned inventions preferably by combining erythritol as the saccharide binder to protein or polysaccharides by heating an aqueous suspension of erythritol and one or other of the two or both together and then allowing it to cool.

By doing this the erythritol is kept in solution or suspension within the food and not allowed to precipitate out. In USPTO published patent application number 20040121062, it was disclosed that saccharides could act as a binder to soybean particles, which contain protein, ultimately in that invention to simulate nuts made of soybeans by mixing soybean particles, a nut flavoring, and a binder comprising a saccharide in a wet flowable form wherein the soybean particles are coated with the binder. Notwithstanding, the simulated nut is still a high calories food, not the ultra low calories food including the suppression of ghrelin that this disclosure seeks to provide, and the polyol of patent application Ser. Nos. 11/301,181 11/358,619 and 11/371,281 is not mentioned in that disclosure, nor are polyols at all. Nevertheless, saccharide binding is what is believed to be the chemical vehicle of patent application number 20040121062 and also of the inventions of patent application Ser. Nos. 11/301,181 11/358,619 and 11/371,281, in these in order to keep erythritol in solution. This disclosure seeks to establish novel means to have erythritol enabled to have a maximum function in suppressing ghrelin by enabling it to reach the small intestine and remain therein in as concentrated a form as is possible.

This invention is designed, among other things, to overcome the deficiencies of previous applications and inventions by disclosing novel new means to enable erythritol to be present in the small intestine in maximum amounts over the shortest period of time to enable the most ghrelin to be suppressed by these means for maximum benefit in weight management.

### SUMMARY OF THE INVENTION

To enable erythritol to be present in maximum concentrations far into the small intestine over the shortest period of time so as to enable the most ghrelin possible to be suppressed by these means for maximum benefit in weight management requires certain things. Novel means are disclosed to incorporate the kind of other food with erythritol that will keep the erythritol in solution within a bolus of ingested food that thus has substantial concentration of the erythritol as it passes into and through the small intestine. Erythritol resists being incorporated with other food in a baking process since it will precipitate out and not stay in solution. On the other hand, erythritol can act as a saccharide binder to protein and the two together enable the erythritol to remain in solution in a confluent manner and not precipitate out of the bolus. To effect this reaction in a basic example, the protein and erythritol must be prepared together with sufficient water being present and the three incorporated into a batter with other food ingredients, to effect the type of pastry or other food being prepared; then the batter's contents subjected to heat to complete the reaction. After a bolus of this prepared food is eaten, it will stay more or less intact as it journeys through the stomach and deep into the intestines until absorption takes place with ghrelin being suppressed by a change in osmolarity provided by the erythritol. Thus if soy margarine 4 oz, whole eggs 3 oz, vanilla extract 1 oz, soy protein isolate 4½ oz, erythritol 9 oz, salt (Koscher) ¼ oz baking soda ¼ oz, cinnamon 1 tsp, nutmeg ½ tsp, chocolate chips 4 oz, and soy nuts 4 oz are blended together and an attempt to bake cookies made, only a non-marketable granular mass will result. This can be remedied by first making an emulsion of the soy protein isolate and erythritol by adding water to the two followed by baking them with the other ingredients so as to affect maximum binding of the erythritol to the protein. Thus if we take one ounce scoops of the batter made of the above ingredients with an appropriate amount of water added and bake cookies at ±350° F., they will lose ¼ ounce of water and we will have ¾ ounce cookies, but the erythritol will stay in solution and not result in hard or granular cookies. Therefore, as a result of the binding process that went on during the baking, the erythritol remains in solution and does not precipitate out. It has the same kind of texture as if sucrose and flour were used although flour by itself can be substituted for the protein.

If the soy-erythritol is to be used to dilute fat in food that is not to be cooked such as with mayonnaise, dips, salad dressings or frozen desserts, the two preferably must be mixed with water and then heated and allowed to cool prior to such use.

Further details and advantages of the present invention are provided in the following more detailed description.

### DETAILED DESCRIPTION OF THE INVENTION, NO DRAWINGS

As cited above, virtually zero calories erythritol helps suppress the appetite-stimulating hormone, ghrelin. Such suppression occurs in part by differential osmolarity in the distal intestine after a meal, and erythritol is the only virtually zero calories sweet substance that will accomplish this, however, taking erythritol by itself will be far less effective because of the dilution effect it must be bound to another substance that a prevents it from immediately going into solution in the stomach fluids. Binding erythritol to protein isolate is the preferred embodiment, but this causes the preferred embodiment to have calories. Fortunately it turns out that erythritol, being the appetite suppressing ingredient, is best present in greater amounts than the protein so more of the preferred embodiment is zero calories than the 4 calories per gram of the protein. Soy protein isolate is the most cost-effective form of protein. The fact that erythritol can help prevent caries formation is another health benefit of this disclosure, although caries prevention by erythritol does occur without its being incorporated into the preferred embodiment.

It would appear obvious that for erythritol to suppress ghrelin to the greatest extent it must be presented to the small intestine in as concentrated form as possible. Baking it into other food accomplishes this purpose, provided in the preferred embodiment sufficient protein is present because the erythritol can be bound to the protein with heat and water and kept in solution without being precipitated out. The preferred embodiment can then be incorporated with other ingredients and rendered a confluent mass with baking that will stay together longer than would be the case if it were in its crystalline state and dissolved directly into the stomach juices becoming diluted immediately. By administering the erythritol in its crystalline form it becomes immersed in the largely aqueous environment of the stomach. If it is bound to protein in an aqueous environment and fixed there by heat such as is the case in baking it after removing it from an appropriate batter, then by it entering the stomach as a cooked bolus, it will remain an insoluble mass in the stomach and await being disintegrated and absorbed as one defined mass of food in the intestines in which the bolus' absorption will occur more all at once instead of being more spread out.

To utilize the preferred embodiment in a marketable bakery product is therefore to combine proportionally as the basic example, soy protein isolate ±4½ oz, with erythritol ±9 oz and ±13½ oz of water and mix. To this can be added whole eggs 3 oz, vanilla extract 1 oz, salt (Koscher) ¼ oz, baking soda ¼ oz, cinnamon 1 tsp, nutmeg ½ tsp, chocolate chips 4 oz, soy nuts 4 oz and finally soy margarine (or butter or other fat) 4 oz and make the ingredients into an appropriate batter and use one ounce scoops of the batter to make individual cookies. Heated at ±350° F. until done will result in ¾ ounce weight-management cookies. The soy nuts and chocolate chips are added into the batter for flavor and can be excluded. If excluded the soy protein isolate and the erythritol combined with water and then with the eggs and soy margarine, etc. will become the basic preferred embodiment and can be extended to any pastry including a crust.

Thus the preferred embodiment is to combine erythritol, soy protein isolate, a fat, preferably a flavorable fat such as butter or margarine, especially soy margarine but even lard will work, to go with the soy protein isolate, eggs or their equivalence, considerable water, equal approximately to the weight of the soy protein isolate combined with the erythritol, plus such ingredients as baking soda, salt and flavorings.

We have found that while all other ingredients can remain steady, including the total amount of protein isolate and erythritol, the ratio of one part by weight of protein isolate and two parts by weight of erythritol is optimum for a number of reasons, including texture formation and sweetness, but they can vary to the point that erythritol precipitates so much that it does not form a confluent mass so as not to be rendered into a workable, tasty batter. Furthermore if sufficiently substantial therein the protein can be part of another food such as soy flour as well as from a dairy source, including whey protein concentrate. Of course whey protein isolate can replace soy protein isolate directly and make the same kind of bakery goods with the same enablement of erythritol to be made into weight-management cookies as described in the preferred embodiment, as well as any means of food preparation as a result of this and its previous disclosures of patent application Ser. Nos. 11/301,181, 11/358,619 and 11/371,281.

Since this disclosure enables ordinary flour and cooking sugar such as brown and table sugar, of the disaccharide sucrose, as well as its components of fructose and glucose and all other hexoses and disaccharides to be replaced by the zero calories, zero glycemic polyol, erythritol, bound to protein in the presence of sufficient water to effect the solution, it is a novel new food as a result of erythritol-protein binding in a cooking process that keeps a substantial amount of erythritol remaining in solution. Since erythritol is prevented from being precipitated out of the process, a specific chemical reaction must take place while cooking, that is different in kind from all previous cooking results. We are able to take a polypeptide or accumulation of polypeptides and bind them to erythritol in the cooking process to enable this new molecule to form long enough to keep the erythritol in solution. Cost-effectively using soy protein isolate is part of the preferred embodiment.

While binding erythritol to protein in the presence of water and heat is the preferred embodiment, it is possible to bind erythritol to dietary complex carbohydrates as patent application Ser. No. 11/301,181, “Z-Trim Combined Directly with Erythritol”, discloses. Published patent application number 20040121062 essentially discloses the binding principle, but that application, as disclosed, does not include erythritol, a sugar alcohol that ordinarily does not stay in solution but readily precipitates out in baking. As taught in all of this inventor's patent applications concerning the use of erythritol in this regard, binding the erythritol to either protein, polysaccharides or both requires that substantial water be used to mix the erythritol with the polysaccharide in the same way as is disclosed above with protein isolate. This novel use of water and erythritol mixed together with the polysaccharide, followed by heat, is what was not apparent to those skilled in the art and what is needed to make erythritol in tasty sizeable amounts available in the small intestine for maximum ghrelin suppression and to take advantage of the virtual zero calories, zero glycemic properties of erythritol for human and animal diets. Of course, the polysaccharide can have protein and even fat included in with it. The important thing is to bind it with erythritol and water followed by heat to keep the erythritol in solution in the resulting bakery product.

Therefore to utilize this embodiment in a marketable bakery product using a dietary polysaccharide or complex carbohydrate instead of protein isolate is to combine proportionally, to use the same basic example, bread flour, cake flour, soy flour, rice flour, oat flour, etc. (the chef's choice) ±4% oz, with erythritol ±9 oz and ±13% oz of water and mix. To this can be added whole eggs 3 oz, vanilla extract 1 oz, salt (Koscher) ¼ oz, baking soda ¼ oz, cinnamon 1 tsp, nutmeg ½ tsp, chocolate chips 4 oz, soy nuts 4 oz and finally soy margarine (or butter or other fat) 4 oz, making the ingredients into an appropriate batter, and use one ounce scoops of the batter to make individual cookies. Heated at ±350° F. until done will result in cookies with this particular batter, but cakes, pies and doughnuts can also have appropriate batters made for them. Again the soy nuts and chocolate chips are added into the batter for flavor and can be excluded. If these are excluded then the polysaccharide and the erythritol combined with water and then with the eggs and fat (margarine, butter, etc.) added will become the basic preferred embodiment when polysaccharides are substituted for protein isolate and can be extended to any pastry including the crust.

Although the invention has been described with reference to particular embodiments and features, other similar embodiments and features may be utilized to obtain similar results. Variations and modifications of erythritol being bound to dietary protein or dietary complex carbohydrates of the present invention will be apparent to one skilled in the art, and the present disclosure is intended to cover all such modifications and equivalents within the scope of the following claims.

I claim

1. Erythritol and a dietary protein isolate are combined with water and then mixed into a batter with other food and heated.

2. The invention according to claim 1 in which the dietary protein is soy protein isolate.

3. The invention according to claim 1 in which the dietary protein is whey protein isolate.

4. The invention according to claim 1 in which the dietary protein is a protein concentrate.

5. The invention according to claim 1 in which ghrelin is suppressed when a bolus of the cooked ingredients has gone substantially into the small intestine and is absorbed thereby, producing differential osmolarity.

6. The invention according to claim 1 in which table sugar and cereal-type flour are eliminated from the batter.

7. The invention according to claim 1 in which table sugar and cereal-type flour are included in the batter.

8. The invention according to claim 1 in which erythrirtol acts as a saccharide binder.

9. The invention according to claim 1 in which dietary polysaccharides are substituted for the protein.
